(19) United States
(12) Patent Application Publication
(10) Pub. No.: US 2026/0015670 A1
(43) Pub. Date: Jan. 15, 2026

(54) NON-INVASIVE CANCER DETECTION METHODS

(71) Applicant: Illumina, Inc., San Diego, CA (US)

(72) Inventors: Mahdi Golkaram, San Diego, CA (US); Frixos Papadopoulos, Cambridge (GB); Fan Song, San Diego, CA (US); Raakhee Vijayaraghavan, San Diego, CA (US); Li Liu, San Diego, CA (US); Jennifer LoCoco, San Diego, CA (US); Chen Zhao, San Diego, CA (US)

(21) Appl. No.: 18/869,229

(22) PCT Filed: Mar. 21, 2024
(86) PCT No.: PCT/US2024/020922

§ 371 Date: Nov. 25, 2024

Related U.S. Application Data
(60) Provisional application No. 63/493,687, filed on Mar. 31, 2023.

(51) Int. Cl. C12Q 1/6886; C12Q 1/6806; G16B 5/00; G16H 50/20

(52) U.S. Cl. CPC C12Q 1/6886; C12Q 1/6806; G16B 5/00; G16H 50/20

(57) ABSTRACT

Method of detecting a cancer and identifying a tissue of origin of cfDNA in a subject involve obtaining a sample from a subject, collecting cfDNA from the sample, generating sequence libraries of ctDNA fragments, analyzing the sequence libraries of the ctDNA fragments, and classifying the sample as having ctDNA fragments from a healthy tissue or a cancer tissue.

### CROSS-REFERENCE TO RELATED APPLICATION

This application claims the benefit of U.S. Provisional Patent Application No. 63/493,687, entitled “NON-INVASIVE CANCER DETECTION METHODS,” filed on Mar. 31, 2023, the disclosure of which is hereby incorporated by reference in its entirety and for all purposes.

### BACKGROUND

### Field

The present disclosure is related to non-invasive cancer detection methods, specifically, the non-invasive cancer detection methods involving multi-modal and targeted analyses of features associated with cell-free DNA fragmentation.

### Description of the Related Art

Sequencing circulating tumor DNA (ctDNA) can provide a unique opportunity for non-invasive tumor profiling and simultaneous assessment of several prognostic and predictive biomarkers, enabling therapy selection.

Effective screening paradigms are crucial to reduce the morbidity and mortality of human cancers worldwide^{1,2}. Recent advances in ctDNA sequencing have provided an opportunity to perform non-invasive cancer detection and may address this unmet need^{3,4}. Several previous studies have highlighted cancer related features in ctDNA that can link ctDNA genome or epigenome to tumor TOO⁵⁻⁹. Namely, the presence of tumor-specific alterations such as somatic chromosomal copy number alterations can suggest tumor derived circulating ctDNA in peripheral blood¹⁰⁻¹². However, only a subset of tumors carries such somatic alterations leading to a significantly low sensitivity of this approach. Besides, higher sequencing coverage is needed to detect these alterations at very low tumor content. Alternatively, genome-wide ctDNA methylation assays targeting individual hypermethylated tumor suppressor genes have proven to be a promising method for early cancer detection^{2,13}. A previous study demonstrated accurate prediction of cancer TOO across >50 cancer types².

In addition, others have shown ctDNA fragmentation patterns carry information that describes chromatin accessibility which can be utilized to trace the tumor TOO¹⁴⁻¹⁶. Nucleosome positioning of human plasma DNA resulted in ctDNA fragments with a characteristic length: a modal size of 166 bp with a series of successive peaks at 10 bp intervals, a consequence of the periodic location of the DNA double helix exposed to DNA endonuclease enzyme. In addition, tumor derived ctDNA fragments tended to be shorter, with a modal size of approximately 143 bp¹⁷.

### SUMMARY

In some embodiments, a method of detecting a cancer in a subject is provided.

In some embodiments, the method of detecting a cancer in a subject method comprises obtaining a sample from a subject, isolating cfDNA from the sample, generating sequence libraries of ctDNA fragments from the isolated cfDNA, analyzing the sequence libraries of the ctDNA fragments to identify a tissue of origin of the ctDNA, thereby detecting the cancer in the subject.

In some embodiments of the method of detecting a cancer in a subject, analyzing the sequence libraries of ctDNA fragments comprises analyzing 5′end 4-mer motifs in the ctDNA fragments.

In some embodiments of the method of detecting a cancer in a subject, analysis of 5′end 4-mer DNA motifs in the ctDNA fragments comprises an unbiased enrichment analysis of one or more motifs associated with a cancer tissue of origin as compared to an expected distribution of the one or more motifs in a healthy tissue of origin.

In some embodiments, the method of detecting a cancer in a subject achieves an AUC of at least 95% in detecting the cancer.

In some embodiments of the method of detecting a cancer in a subject, analyzing the sequence libraries of the ctDNA fragments comprises performing a window protection score (WPS) analysis.

In some embodiments of the method of detecting a cancer in a subject, the window protection score analysis comprises determining a ratio of a number of endpoints of the ctDNA fragments within a 120 bp ctDNA fragment size window to a number of fragments completely spanning the 120 bp ctDNA fragment size window.

In some embodiments of the method of detecting a cancer in a subject, a high WPS value as compared to a threshold indicates an increased protection of the ctDNA from digestion.

In some embodiments of the method of detecting a cancer in a subject, a low WPS value as compared to a threshold indicates a decreased protection of the ctDNA from digestion.

In some embodiments, the method of detecting a cancer in a subject achieves an AUC of at least 95% in detecting the cancer.

In some embodiments of the method of detecting a cancer in a subject, analyzing the sequence libraries of the ctDNA fragments comprises performing a genome wide fragmentation length distribution (GWFLD) analysis.

In some embodiments of the method of detecting a cancer in a subject, the GWFLD analysis comprises determining a ratio of a number of ctDNA fragments that range in size from greater than 100 to less than 150 bp to a number of ctDNA fragments that range in size from greater than 151 to less than 220 bp within a 5 Mbp ctDNA fragment size window.

In some embodiments of the method of detecting a cancer in a subject, the method achieves an AUC of more than 90% in detecting the cancer.

In some embodiments of the method of detecting a cancer in a subject, generating sequence libraries of ctDNA fragments comprises targeted enrichment of 100 or more, 200 or more, 300 or more, 400 or more, 500 or more genomic regions.

In some embodiments, a method of identifying a tissue of origin of cfDNA in a subject is provided.

In some embodiments, the method of identifying a tissue of origin of cfDNA in a subject comprises obtaining a sample from a subject, isolating cfDNA from the sample, generating sequence libraries of ctDNA fragments from the cfDNA, aligning paired-end reads of the ctDNA fragments, analyzing 5′end 4-mer motifs, window protection score (WPS), and genome wide fragmentation length distribution (GWFLD) of the ctDNA fragments, generating independent data models for 5′end 4-mer motifs, WPS, and GWFLD, generating an ensemble model of the independent models from the independent data models using a machine learning process, and classifying the sample as comprising ctDNA fragments from a healthy tissue or a cancer tissue based on the ensemble model.

In some embodiments, the method of identifying a tissue of origin of cfDNA in a subject provides at least 80% sensitivity at 99.9% specificity in classifying the sample as comprising ctDNA from a healthy tissue or a cancer.

In some embodiments, the method of identifying a tissue of origin of cfDNA in a subject provides at least 80% accuracy in predicting a tissue of origin of the ctDNA fragments.

In some embodiments of the method of identifying a tissue of origin of cfDNA in a subject, generating sequence libraries of ctDNA fragments comprises targeted enrichment of 100 or more, 200 or more, 300 or more, 400 or more, 500 or more genomic regions.

In some embodiments, a method of determining a presence of cancer in a patient is provided.

In some embodiments, the method of determining a presence of cancer in a patient the method comprises isolating cfDNA from samples from a plurality of subjects, generating sequence libraries of ctDNA fragments from the isolated cfDNA, aligning paired-end reads of the ctDNA fragments, analyzing 5′end 4-mer motifs, window protection score (WPS), and genome wide fragmentation length distribution (GWFLD) of the ctDNA fragments, generating independent models for 5′end 4-mer motifs, WPS, and GWFLD, generating an ensemble model of the independent models using a machine learning process from the independent models, applying the ensemble model to sequence libraries of ctDNA fragments for a sample obtained from the patient, and determining whether the patient has cancer based on the application of the ensemble model.

In some embodiments of the method of determining a presence of cancer in a patient, generating sequence libraries of ctDNA fragments comprises targeted enrichment of 100 or more, 200 or more, 300 or more, 400 or more, 500 or more genomic regions.

In some embodiments of the methods herein, the sample is blood.

In some embodiments of the methods herein, the cancer is one or more of lung cancer, liver cancer, renal cancer, breast cancer, glioma, or colorectal cancer.

In some embodiments of the methods herein, wherein generating sequence libraries of ctDNA fragments comprises enriching, isolating, and sequencing a subset of genes or genomic region of interest.

In some embodiments of the methods herein, wherein enriching, isolating, and sequencing a subset of genes or regions of a genome comprises capturing a subset of genes or genomic region of interest by hybridization of ctDNA fragments to probes that are specific for the subset of genes or genomic region of interest.

### BRIEF DESCRIPTION OF THE DRAWINGS

FIG. 1 shows an embodiment of a schematic of a study design.

FIGS. 2a-i show the results of an end motif analysis, which is a cancer-type specific enrichment of distinct motifs linking fragmentation pattern to tissue of origin. FIG. 2a is a bar graph showing motif z-score for lung cancer compared to normal tissue. FIG. 2b is a bar graph showing motif z-score for breast cancer compared to normal tissue. FIG. 2c is a bar graph showing motif z-score for colorectal cancer compared to normal tissue. FIG. 2d is a bar graph showing motif z-score for liver cancer compared to normal tissue. FIG. 2e is a bar graph showing motif z-score for glioma compared to normal tissue. FIG. 2f is a bar graph showing pan-cancer motif z-score compared to normal tissue. For each cancer type, top 4 representative motifs with the most significant enrichment are shown. Wilcox test P reported after FDR correction. FIG. 2g is a plot showing the motif diversity score (MDS) for various cancers. FIG. 2h is a plot showing the frequency distribution of DNA fragments based on window protection score (WPS) analysis for various cancers. Fragment length distribution of all samples are shown and colored based on TOO. FIG. 2i is a plot showing the ratio of the number of small to large fragments based on genome wide fragmentation length distribution (GWFLD) analysis used to classify samples to cancer versus non-cancer.

FIGS. 3a-i show the results of an embodiment of machine learning based classification of cancer samples. FIG. 3a is a plot showing an analysis based on a model of a 2-fold cross-validation of a distribution of 5′ end motifs (4-mer). FIG. 3b is a plot showing an analysis based on a model of a 2-fold cross-validation of a distribution of window protection score (WPS). FIG. 3c is a plot showing an analysis based on a model of a 2-fold cross-validation of a distribution of genome wide fragmentation length. FIG. 3d is a plot showing an analysis based on a model of a 5-fold cross-validation of a distribution of 5′ end motifs (4-mer). FIG. 3e is a plot showing an analysis based on a model of a 5-fold cross-validation of a distribution of window protection score (WPS). FIG. 3f is a plot showing an analysis based on a model of a 5-fold cross-validation of a distribution of genome wide fragmentation length. FIG. 3g is plot showing in silico down sampling of ctDNA sequencing reads to assess the sensitivity of the 5′ end motifs (4-mer) distribution model as a function of total number of reads used for classification. FIG. 3h is plot showing in silico down sampling of ctDNA sequencing reads to assess the sensitivity of the window protection score (WPS) distribution model as a function of total number of reads used for classification. FIG. 3g is plot showing in silico down sampling of ctDNA sequencing reads to assess the sensitivity of the genome wide fragmentation length distribution (GWFLD) model as a function of total number of reads used for classification.

FIGS. 4a-c show the results of an embodiment of an ensemble classifier, which can integrate multimodal fragmentomics data for cancer screening. FIG. 4a shows the results of the overall concordance of the three models comparing predicted and true values using LOO CV. FIG. 4b shows performance results of an ensemble classifier after 2-fold CV as well as validation on a completely independent set of ccRCC samples. FIG. 4c shows the results of ensemble classifier's prediction of TOO.

### DETAILED DESCRIPTION

Embodiments relate to the discovery that the distribution of end motifs in ctDNA can be used to distinguish tumor vs non-tumor derived ctDNA and can be utilized for non-invasive cancer detection. The fragmentation pattern of targeted ctDNA sequencing was reviewed and evaluated to determine whether different fragmentation features could be used for non-invasive cancer detection. Then, using an ensemble-based machine learning system and method, it was shown that combining distinct fragmentation features improved cancer vs non-cancer classification and prediction of the tumor tissue of origin (TOO).

Sequencing circulating tumor DNA (ctDNA) can provide a unique opportunity for non-invasive tumor profiling and simultaneous assessment of several prognostic and predictive biomarkers, enabling therapy selection. However, the utility of ctDNA sequencing in cancer detection and identifying the tissue of origin is less established. As discussed below, ctDNA of 509 participants including cancer patients across tumor types and healthy donors was collected from multiple institutions. Targeted ctDNA sequencing was performed using TruSight™ Oncology 500 (TSO500) ctDNA assay. Motif analysis, window protection score (WPS), and genome wide fragmentation length distribution (GWFLD) were independently linked to TOO. Finally, using machine learning, these features were combined into an ensemble model to classify samples into healthy or cancer TOO. Using a random forest model and 5-fold cross validation, 5′-end motif model. WPS model, and GWFLD model independently achieved area under the curve (AUC) of 98.3% (CI: 96.3%-100%), 98.7% (CI: 96.6%-100%), and 94.6% (CI: 90.3%-99%) in detecting cancer from blood samples. Finally, an ensemble machine learning model achieved >800% sensitivity at 99.9% specificity in detecting cancer vs non-cancer samples with ˜83% TOO prediction accuracy.

Using targeted sequencing of ctDNA collected from 509 donors including healthy and cancer patients, a link was demonstrated between the origin and the fragmentation features of plasma ctDNA including end motif, WPS, and GWFLD. As previously suggested, a strong enrichment was observed of smaller fragments in ctDNA derived from cancer patients. Moreover, MDS—a measure of diversity of ctDNA end motifs—trended higher in cancer samples compared to ctDNA from healthy donors although this observation varied across different cancer types. By building three independent machine learning models using these fragmentomics features, it was shown that fragmentation patterns of ctDNA, even within a limited genomic range, can be informative of the origin of ctDNA.

Since fragmentation features can be observed in methylation sequencing of circulating ctDNA, the disclosed ensemble learning framework can be recruited to boost the accuracy of methylation based ctDNA cancer detection by combining independent classifiers each of which encompass distinct biological observations. Other fragmentation features, such as jagged end length of ctDNA²², preferred end coordinate^{23,24}, and ctDNA integrity^{20,25,26} may also be used to improve the ability to predict cancer TOO from circulating plasma DNA. Beyond cancer detection, embodiments may be used for non-invasive prenatal testing, autoimmune diseases, as well as treatment monitoring by measuring minimal residual disease (MRD).

Embodiments of the present invention leveraged targeted sequencing of ctDNA using the TruSight Oncology 500 ctDNA assay. However, aspects are not limited to this particular assay or device. In some embodiments, the disclosed approach has the advantage of ultra-high coverage (>1300×) covering 523 cancer related genes (˜1.3 Mbp coding region) and thereby, detecting several genetic alterations even at extremely low tumor fractions. This allows clinicians to efficiently perform comprehensive genomic profiling of liquid biopsies within the same assay which can serve as a guide towards therapy selection in the follow-up practice. In addition, it was shown that targeted sequencing using TruSight Oncology 500 ctDNA required a substantially lower amount of DNA sequencing suggesting a cost-effective alternative to whole genome sequencing of ctDNA.

In some embodiments, generating sequence libraries of ctDNA fragments comprises enriching, isolating, and sequencing a subset of genes or genomic region of interest. In some embodiments, a sample can be enriched for ctDNA fragments of interest (“target ctDNA sequences”). For example, the target ctDNA sequences comprise a subset of genes or regions of a genome that are isolated and sequenced. In some embodiments, enrichment of target ctDNA sequences is achieved by capturing regions of a genome by hybridization to target-specific probes (e.g., target ctDNA sequence-specific probes). Thus, in some embodiments, enriching, isolating, and sequencing a subset of genes or regions of a genome comprises capturing a subset of genes or genomic region of interest by hybridization of ctDNA fragments to probes that are specific for the subset of genes or genomic region of interest. The target-specific probes can be used to physically separate target ctDNA sequences that have hybridized to the target-specific probes from all other DNA. Any genomic sequences that do not hybridize to the target-specific probes can be removed by, for example, by washing away in solution. For example, some methods of enrichment of target ctDNA sequences can utilize biotinylated probes, which are then isolated by magnetic separation with streptavidin-coated magnetic particles.

Enriching a nucleic acid of interest (e.g., target ctDNA sequences), or a fragment thereof, such as enriching DNA in a sample, may include any suitable enrichment technique or combination of techniques. In some embodiments, enrichment of a nucleic acid of interest (e.g., target ctDNA sequences) may include enrichment through molecular inversion probes, in solution capture, pulldown probes, bait sets, standard PCR, multiplex PCR, hybrid capture, endonuclease digestion, DNase I hypersensitivity, and selective circularization. Enrichment can be achieved through negative selection of nucleic acids by eliminating undesired material. This sort of enrichment includes ‘footprinting’ techniques or ‘subtractive’ hybrid capture. During the former, the target sample is safe from nuclease activity through the protection of protein or by single and double stranded arrangements. During the latter, nucleic acids that bind ‘bait’ probes are eliminated.

In some embodiments, enriching includes amplification of the DNA using target-specific primers. In some embodiments, amplification occurs after another form of enrichment. In some such embodiments, amplification comprises PCR amplification or genome-wide amplification.

In some embodiments, the sequence library of ctDNA fragments can comprise an unenriched library that is representative of a whole genome. Some embodiments include a step of enrichment of a portion of the whole genome. For example, some such embodiments include targeted enrichment of less than 100% of a human genome, such as less than 1%, 2%, 3%, 4%, 5%, 6%, 7%, 8%, 9%, or less than 10% of the entire genome. In some embodiments, targeted enrichment is of less 10%/6, 20%, 30%, 40%, 50%, 600%, 70%, 80%, or less than 90% of the whole genome. In some embodiments, generating sequence libraries of ctDNA fragments comprises targeted whole exome enrichment, partial exome enrichment, or a combination thereof.

In some embodiments, generating sequence libraries of ctDNA fragments comprises targeted enrichment of 10 or more, 20 or more, 30 or more, 40 or more, 50 or more, 60 or more, 70 or more, 80 or more, 90 or more, 100 or more, 200 or more, 300 or more, 400 or more, 500 or more genomic regions. In some such embodiments, the genomic regions include exons and introns of genes of interest. In some such embodiments, the genomic regions include exons of genes of interest. In some such embodiments, the genomic regions include introns of genes of interest. Non-limiting examples of genes of interest include cancer-related genes.

Non-limiting examples of cancer-related genes include breast cancer-related genes (e.g., ATM, BARD1, BRCA1, BRCA2, BRIP1, CHEK2, CDH1, NF1, PALB2, PTEN, RAD51C, RAD51D, STK11, TP53), colorectal cancer-related genes (e.g., APC, EPCAM, MLH1, MSH2, MSH6, PMS2, CHEK2, PTEN, STK11, TP53, MUTYH), endometrial cancer-related genes (e.g., BRCA1, EPCAM, MLH1, MSH2, MSH6, PMS2, PTEN, STK11), fallopian tube, ovarian, primary peritoneal cancer-related genes (e.g., ATM, BRCA1, BRCA2, BRIP1, EPCAM, MLH1, MSH2, MSH6, NBN, PALB2, RAD51C, RAD51D), gastric cancer-related genes (e.g., APC, CDH1, STK11, EPCAM, MLH1, MSH2, MSH6, PMS2), melanoma-related genes (e.g., BAP1 (especially uveal melanoma), BRCA2 CDK4, CDKN2A, PTEN, TP53), pancreatic cancer-related genes (e.g., ATM, BRCA1, BRCA2, CDKN2A, EPCAM, MLH1, MSH2, MSH6, PALB2, STK11, TP53), prostate cancer-related genes (e.g., ATM, BRCA1, BRCA2, CHEK2, HOXB13, PALB2, EPCAM, MLH1, MSH2, MSH6, PMS2).

In some embodiments, a genome can is a human genome. In some embodiments, a genome is a non-human genome.

In some embodiments, such as those described in the non-limiting Examples below, a panel of targeted enrichment probes is used to enrich 500 or more cancer related genes. An exemplary panel of such enrichment probes used in the embodiments presented herein can include a TruSight Oncology 500 assay available from Illumina, Inc. (San Diego, CA) covering 523 cancer related genes (˜1.3 Mbp coding region). Other examples of enrichment panels that could be used in embodiments presented herein include those found in assays such as FoundationOne Assay and FoundationOne CDx Assay (Foundation Medicine, Inc.), Oncomine Precision assay, Oncomine Focus assay, Oncomine Comprehensive assay, Oncomine tumor-specific panels and related Oncomine assays (Thermo Fisher Scientific, Inc.), Guardant360 assays (Guardant Health, Inc.), Tempus TO (tumor origen), Tempus HRD, Tempus xT, Tempus xF, Tempus xE, and Tempus xG assays (Tempus), and other similar enrichment panel based assays.

### Methods of Detecting a Cancer

In some embodiments, a method of detecting a cancer in a subject is provided.

In some embodiments, the method of detecting a cancer in a subject comprises obtaining a sample from a subject, isolating cfDNA from the sample, generating sequence libraries of ctDNA fragments from the isolated cfDNA, analyzing the sequence libraries of the ctDNA fragments to identify a tissue of origin of the ctDNA, thereby detecting the cancer in the subject.

As used herein, “circulating free DNA” (cfDNA) refers to degraded DNA fragments released from injured, diseased, traumatized, inflamed, or septic tissue/organ. In some embodiments, the cfDNA fragments range in size from about 50 bp to about 200 bp. In some embodiments, the cfDNA fragments range in size from about 50 bp to about 100 bp. The cfDNA fragments range in size from about 100 bp to about 150 bp. In some embodiments, the cfDNA fragments range in size from about 150 bp to about 200 bp. In some embodiments, the cfDNA fragments are released into the blood and/or other body fluids. Nonlimiting examples of cfDNA include circulating tumor DNA (ctDNA), cell-free mitochondrial DNA (cf mtDNA), and cell-free fetal DNA (cffDNA).

In some embodiments of the method of detecting a cancer in a subject, analyzing the sequence libraries of ctDNA fragments comprises analyzing 5′end 4-mer motifs in the ctDNA fragments.

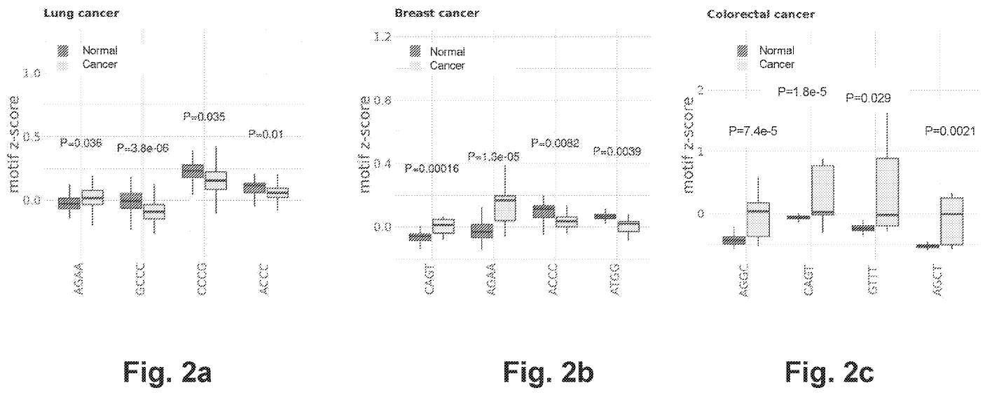

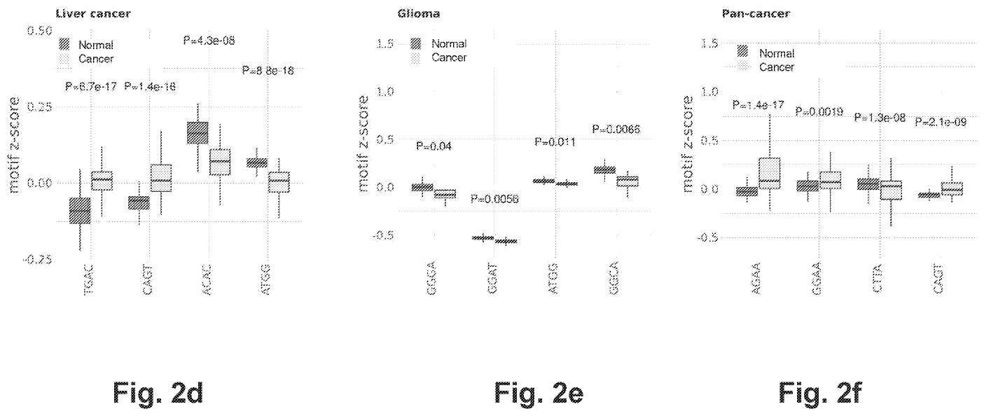

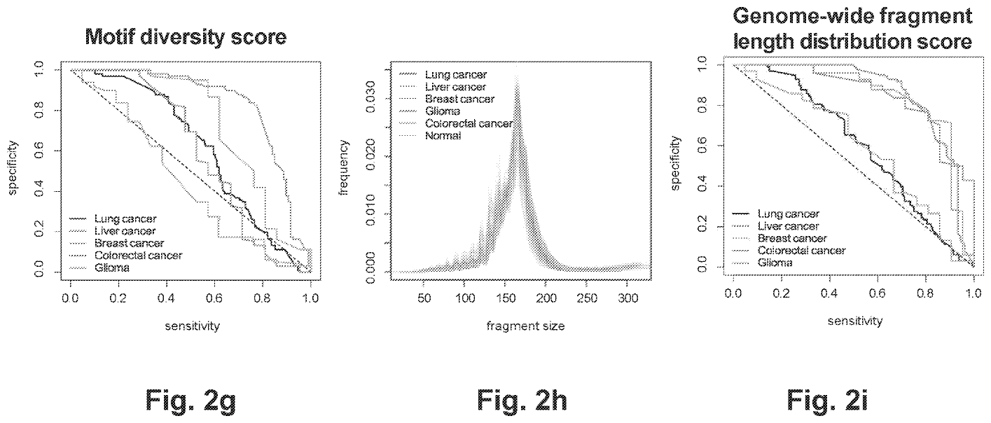

In some embodiments, certain 5′end 4-mer motifs are significantly enriched in a particular cancer tissue/organ as compared to normal tissue/organ. Non-limiting examples of 5′end 4-mer motifs that are enriched in particular cancers are provided in Table 1 (See, FIGS. 2a-f).

TABLE 1

| Motif | Cancer type | | | | | |
|---|---|---|---|---|---|---|
| AGAA | Lung | Breast | | | | Pan- |
| | | | | | | cancer |
| GCCC | Lung | | | | | |
| CCCG | Lung | | | | | |
| ACCC | Lung | Breast | | | | |
| CAGT | | Breast | Colo- | Liver | | Pan- |
| | | | rectal | | | cancer |
| ATGG | | Breast | | Liver | Glioma | |
| AGGC | | | Colo- | | | |
| | | | rectal | | | |
| GTTT | | | Colo- | | | |
| | | | rectal | | | |
| AGCT | | | Colo- | | | |
| | | | rectal | | | |
| TGAC | | | | Liver | | |
| ACAC | | | | Liver | | |
| GGGA | | | | | Glioma | |
| GGAT | | | | | Glioma | |
| GGCA | | | | | Glioma | |
| GGAA | | | | | | Pan- |
| | | | | | | cancer |
| CTTA | | | | | | Pan- |
| | | | | | | cancer |

In some embodiments, the enrichment of the 5′end 4-mer motifs in the cancers is expressed as motif z-score (See, FIGS. 2a-f). as used herein, a “Z-score” is a numerical measurement that describes a motif's relationship to the mean of a group of motifs. Z-score is measured in terms of standard deviations from the mean. If a Z-score is 0, it indicates that the motif's score is identical to the mean score for the motif.

In some embodiments, a 5′end 4-mer motif that is significantly enriched in a particular cancer tissue/organ is n₁n₂n₃n₄, wherein n₁ is A, T, G, or C, n₂ is A, T, G, or C, n₃ is A, T, G, or C, and n₄ is A, T, G, or C.

In some embodiments of the method of detecting a cancer in a subject, analysis of 5′end 4-mer DNA motifs in the ctDNA fragments comprises an unbiased enrichment analysis of one or more motifs associated with a cancer tissue of origin as compared to an expected distribution of the one or more motifs in a healthy tissue of origin.

In some embodiments of the method of detecting a cancer in a subject, the method achieves an AUC of at least 95% in detecting the cancer. In some embodiments of the method of detecting a cancer in a subject, the method achieves an AUC of 95, 95.5, 96, 96.5, 97, 97.5, 98, 98.5, 98.6, 98.7, 98.8, 98.9, or 99%/6 in detecting the cancer. In some embodiments of the method of detecting a cancer in a subject, the method achieves an AUC of 98.3% (CI: 96.3%-100%) in detecting the cancer.

In some embodiments of the method of detecting a cancer in a subject, analyzing the sequence libraries of the ctDNA fragments comprises performing a window protection score (WPS) analysis. As used herein, “window protection score” (WPS) is calculated by determining a ratio of a number of endpoints of DNA fragments (e.g., ctDNA fragments) within a 120 bp window to the number of DNA fragments (e.g., ctDNA fragments) completely spanning the 120 bp window. A high WPS value indicates an increased protection of DNA from digestion (e.g., by nucleases). In contrast, a low WPS value indicates that DNA is unprotected from digestion.

In some embodiments of the method of detecting a cancer in a subject, the window protection score analysis comprises determining a ratio of a number of endpoints of the ctDNA fragments within a 120 bp ctDNA fragment size window to a number of fragments completely spanning the 120 bp ctDNA fragment size window.

In some embodiments of the method of detecting a cancer in a subject, a high WPS value as compared to a threshold indicates an increased protection of the ctDNA from digestion.

In some embodiments of the method of detecting a cancer in a subject, a low WPS value as compared to a threshold indicates a decreased protection of the ctDNA from digestion.

In some embodiments of the method of detecting a cancer in a subject, the method achieves an AUC of at least 95% in detecting the cancer. In some embodiments of the method of detecting a cancer in a subject, the method achieves an AUC of 95, 95.5, 96, 96.5, 97, 97.5, 98, 98.5, 98.6, 98.7, 98.8, 98.9, or 99% in detecting the cancer. In some embodiments of the method of detecting a cancer in a subject, the method achieves an AUC of 98.7% (CI: 96.6%-100%) in detecting the cancer.

In some embodiments of the method of detecting a cancer in a subject, analyzing the sequence libraries of the ctDNA fragments comprises performing a genome wide fragmentation length distribution (GWFLD) analysis.

In some embodiments of the method of detecting a cancer in a subject, genome wide fragmentation length distribution analysis comprises determining a ratio of a number of DNA fragments (e.g., ctDNA fragments) that range in size from greater than 100 to less than 150 bp to a number of DNA fragments (e.g., ctDNA fragments) that range in size from greater than 151 to less than 220 bp within a 5 Mbp DNA fragment size window.

In some embodiments of the method of detecting a cancer in a subject, genome wide fragmentation length distribution analysis comprises determining a ratio of a number of ctDNA fragments that range in size from greater than 100 to less than 150 bp to a number of ctDNA fragments that range in size from greater than 151 to less than 220 bp within a 5 Mbp ctDNA fragment size window.

In some embodiments of the method of detecting a cancer in a subject, the method achieves an AUC of more than 90% in detecting the cancer. In some embodiments of the method of detecting a cancer in a subject, the method achieves an AUC of 91, 91.5, 92, 92.5, 93, 93.5, 94, 94.5, 95, 95.5, 96, 96.5, 97, 97.5, 98, 98.5, or 99% in detecting the cancer. In some embodiments of the method of detecting a cancer in a subject, the method achieves an AUC of 94.6% (CI: 90.3/6-99%) in detecting the cancer.

Method of Identifying a Tissue of Origin of cfDNA

In some embodiments, a method of identifying a tissue of origin of cfDNA in a subject is provided.

In some embodiments, the method of identifying a tissue of origin of cfDNA in a subject the method comprises obtaining a sample from a subject, isolating cfDNA from the sample, generating sequence libraries of ctDNA fragments from the cfDNA, aligning paired-end reads of the ctDNA fragments, analyzing 5′end 4-mer motifs, window protection score (WPS), and genome wide fragmentation length distribution (GWFLD) of the ctDNA fragments, generating independent data models for 5′end 4-mer motifs, WPS, and GWFLD, generating an ensemble model of the independent models from the independent data models using a machine learning process, and classifying the sample as comprising ctDNA fragments from a healthy tissue or a cancer tissue based on the ensemble model.

In some embodiments of the method of identifying a tissue of origin of cfDNA in a subject, the method provides at least 80% sensitivity at 99.9% specificity in classifying the sample as comprising ctDNA from a healthy tissue or a cancer. In some embodiments of the method of identifying a tissue of origin of cfDNA in a subject, the method provides 80, 81, 82, 83, 84, 85, 86, 87, 88, 89, 90, 91, 92, 93, 94, 95, 96, 97, 98, or 99% sensitivity at 99.9% specificity in classifying the sample as comprising ctDNA from a healthy tissue or a cancer.

In some embodiments of the method of identifying a tissue of origin of cfDNA in a subject, the method provides at least 80% accuracy in predicting a tissue of origin of the ctDNA fragments. In some embodiments of the method of identifying a tissue of origin of cfDNA in a subject, the method provides 80, 81, 82, 83, 84, 85, 86, 87, 88, 89, 90, 91, 92, 93, 94, 95, 96, 97, 98, or 99% accuracy in predicting a tissue of origin of the ctDNA fragments.

### Method of Determining a Presence of Cancer

In some embodiments, a method of determining a presence of cancer in a patient is provided.

In some embodiments, the method of determining a presence of cancer in a patient comprises isolating cfDNA from samples from a plurality of subjects, generating sequence libraries of ctDNA fragments from the isolated cfDNA, aligning paired-end reads of the ctDNA fragments, analyzing 5′end 4-mer motifs, window protection score (WPS), and genome wide fragmentation length distribution (GWFLD) of the ctDNA fragments, generating independent models for 5′end 4-mer motifs, WPS, and GWFLD, generating an ensemble model of the independent models using a machine learning process from the independent models, applying the ensemble model to sequence libraries of ctDNA fragments for a sample obtained from the patient, and determining whether the patient has cancer based on the application of the ensemble model.

In some embodiments, the independent model generated for the 5′end 4-mer motifs is referred to as Independent Model 1. In some embodiments, the independent model generated for the WPS is referred to as Independent Model 2. In some embodiments, the independent model generated for the GWFLD is referred to as Independent Model 3. In some embodiments, the ensemble model is generated using machine learning process using at least two independent models. As used herein, “machine learning” is based on algorithms that build a model based on sample data (known as training data) with known parameters. The model can then be used to make predictions or decisions on test data without being explicitly programmed to do so. In some embodiments, the ensemble model is generated using machine learning process using a combination of Independent Models 1 and 2. In some embodiments, the ensemble model is generated using machine learning process using a combination of Independent Models 1 and 3. In some embodiments, the ensemble model is generated using machine learning process using a combination of Independent Models 2 and 3. In some embodiments, the ensemble model is generated using machine learning process using a combination of Independent Models 1, 2, and 3.

In some embodiments, the independent models for 5′end 4-mer motifs, WPS, and GWFLD achieved high sensitivity and specificity in predicting cancer samples. In some embodiments, the independent models use 2-fold to 10-fold cross validation. In some embodiments, the independent models use 2-fold cross validation. In some embodiments, the independent models use 3-fold cross validation. In some embodiments, the independent models use 4-fold cross validation. In some embodiments, the models use 5-fold cross validation. In some embodiments, the independent models use 6-fold cross validation. In some embodiments, the independent models use 7-fold cross validation. In some embodiments, the independent models use 8-fold cross validation. In some embodiments, the independent models use 9-fold cross validation. In some embodiments, the independent models use 10-fold cross validation.

In some embodiments, the number of reads for each of the independent models ranges from about 4×10⁶ to about 0.04×10⁶. In some embodiments, the number of reads for each of the independent models ranges from about 4×10⁶ to about 0.4×10⁶. In some embodiments, the number of reads for each of the independent models ranges from about 0.4×10⁶ to about 0.04×10⁶. In some embodiments, the number of reads for each of the independent models ranges from about 8×10⁶ to about 0.08×10⁶. In some embodiments, the number of reads for each of the independent models ranges from about 8×10⁶ to about 0.8×10⁶. In some embodiments, the number of reads for each of the independent models ranges from about 0.8×10⁶ to about 0.08×10⁶. In some embodiments, the number of reads for each of the independent models ranges from about 2×10⁶ to about 0.02×10⁶. In some embodiments, the number of reads for each of the independent models ranges from about 2×10⁶ to about 0.2×10⁶. In some embodiments, the number of reads for each of the independent models ranges from about 0.2×10⁶ to about 0.02×10⁶.

### Additional Embodiments

In some embodiments of the methods disclosed herein, the sample is blood. In some embodiments of the methods disclosed herein, the sample is a tissue biopsy, plasma, serum, lymph, semen, saliva, sweat, tears, cerebrospinal fluid, synovial fluid, and amniotic fluid.

In some embodiment, the subject is a human. In some embodiments, the methods provided herein can be extrapolated to other organisms. Non-limiting examples include, non-human primates, rats, mice, dogs, cats, guinea pigs, and cattle.

In some embodiments of the methods disclosed herein, the cancer is one or more of lung cancer, liver cancer, renal cancer, breast cancer, glioma, or colorectal cancer.

Non-limiting examples of cancers include breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, or any neoplasm associated with brain including, but not limited to, astrocytomas (e.g., pilocytic astrocytoma, diffuse astrocytoma, anaplastic astrocytoma, and brain stem gliomas), glioblastomas (e.g., glioblastomas multiforme), meningioma, other gliomas (e.g., ependymomas, oligodendrogliomas, and mixed gliomas), and other brain tumors (e.g., pituitary tumors, craniopharyngiomas, germ cell tumors, pineal region tumors, medulloblastomas, and primary CNS lymphomas). In some embodiments, the cancer is related to one or more types of cancers provided herein.

In some embodiments, the cancer is resistant to one or more anticancer drugs. In some embodiments, the cancer is sensitive to one or more anticancer drugs. In some embodiments, the cancer is sensitive to one or more anticancer drugs but resistant to other one or more anticancer drugs.

In some embodiments, the subject is under treatment with one or more anticancer drugs. In some embodiments, the subject is not under treatment with one or more anticancer drugs. In some embodiments, the cancer has relapsed following remission by prior treatment with one or more anticancer drugs.

Non-limiting examples of anticancer drugs include cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

In some embodiments, the subject may be undergoing one or more other anticancer therapies. Non-limiting examples include surgery, radiation therapy, chemotherapy, immunotherapy, targeted therapy, hormone therapy, stem cell transplant, cytokine therapy, gene therapy, cell therapy, phototherapy, thermotherapy, and sound therapy.

### EXAMPLES

The following examples are non-limiting and other variants within the scope of the art also contemplated.

### Example 1

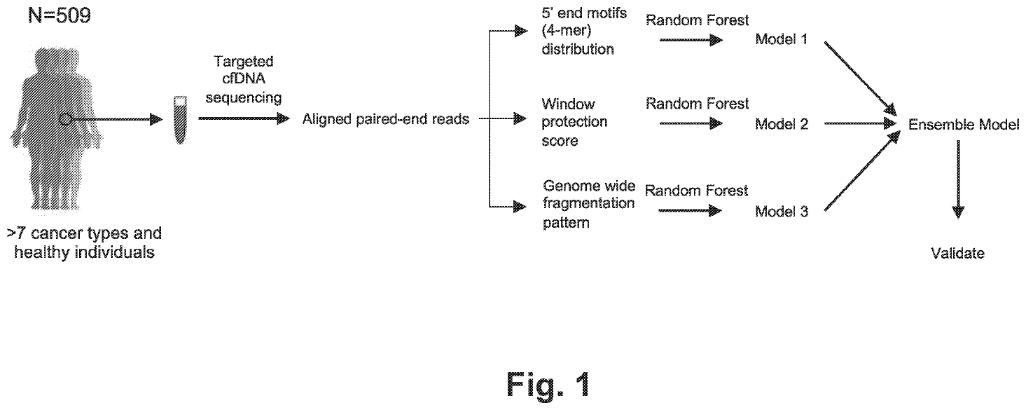

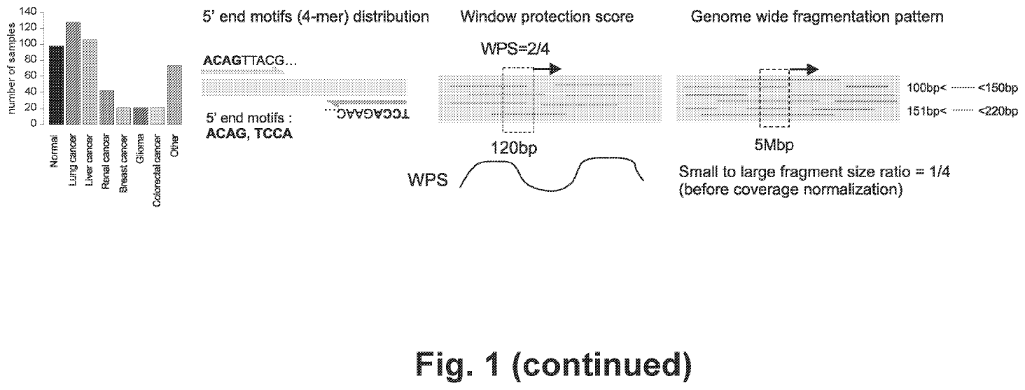

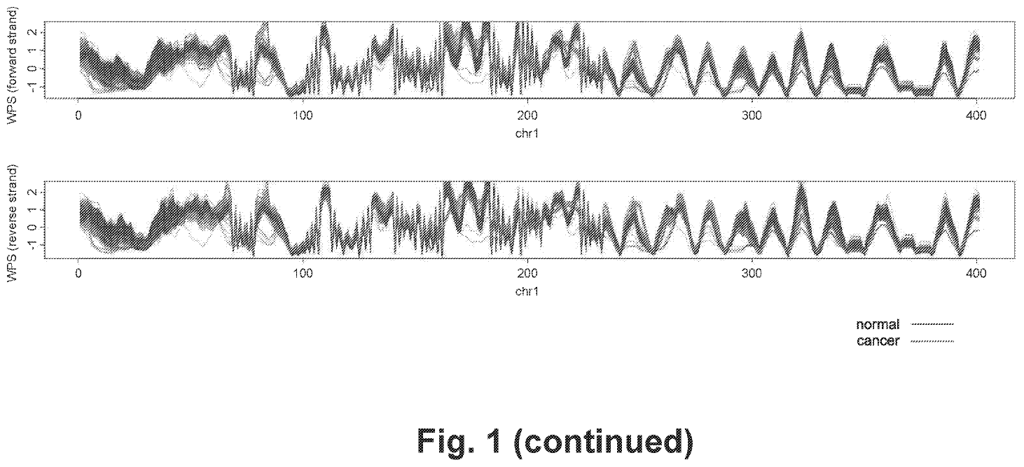

Blood samples from 509 healthy donors and cancer patients were collected and targeted ctDNA sequencing was performed using TSO500 assay. Paired-end aligned reads were analyzed and fragmentation features were extracted and used as features of three separate random forest machine learning models as well as a single ensemble model integrating the three fragmentomics models. CtDNA from the 509 blood samples from cancer patients as well as healthy donors were collected from multiple institutions. To evaluate whether fragmentomics can link targeted ctDNA fragmentation pattern to ctDNA origin, ctDNA library preparation was performed and sequencing using TruSight Oncology 500 ctDNA assay (Illumina Inc., San Diego, CA) and analyzed the sequencing data to evaluate several fragmentation features (FIG. 1). Among cancer types, patients were mainly diagnosed with lung cancer, liver cancer, clear cell renal cell carcinoma (ccRCC), breast cancer, colorectal cancer, and glioma. As described earlier, WPS, GWFLD and motifs were measured for each sample. It was first assessed whether WPS of samples from cancer patients differed from healthy donors across the genome. As expected, a pan-cancer estimation of WPS reflected distinct over and under representation of ctDNA fragment break points at individual genomic loci suggesting targeted ctDNA derived WPS can indicate traces of tumor in peripheral blood (FIG. 1).

### Example 2

Next, whether certain motifs were enriched in different cancer types was investigated. An unbiased motif enrichment analysis of each cancer type compared to the expected distribution (as obtained by motif frequency of healthy individuals), demonstrated highly frequent motifs that vary among different cancer types. Notably, liver and breast cancer exhibited the strongest motif enrichment compared to others while glioma had the lowest effect size (FIGS. 2a-f). Most motifs showed cancer type specific enrichment even though pan-cancer enrichment in several 4-mers was also observed including AGAA and CAGT. Previously, Jiang et al. is reported that a high motif diversity score (MDS) as measured by normalized Shannon entropy can indicate a higher variability of plasma DNA molecules with different end motifs such that MDS of hepatocellular carcinoma were found to be significantly higher than healthy control samples. The same study reported an AUC=0.85 when MDS was used to classify samples into cancer versus non-cancer using MDS compared to fragment size distribution (AUC=0.74). As expected, similar analysis in the disclosed cohort confirmed this finding even though the classification accuracy widely varied across different tumor types with highest accuracy in liver cancer compared to AUC=0.5 in glioma (FIG. 2g). Conversely, using fragment size distribution to distinguish cancer versus non-cancer samples in the disclosed cohort (as measured by the fraction of the reads with fragment size larger than 150 bp across the targeted genome) achieved similar performance to MDS-based classification although the classification of glioma and lung cancer samples showed relatively poor performance when compared to liver and breast cancer (FIGS. 2h, i).

### Example 3

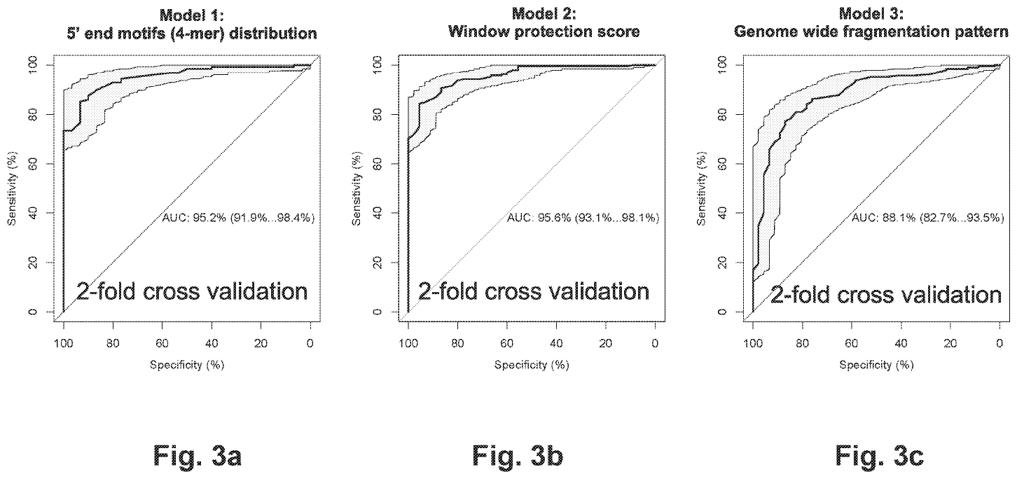

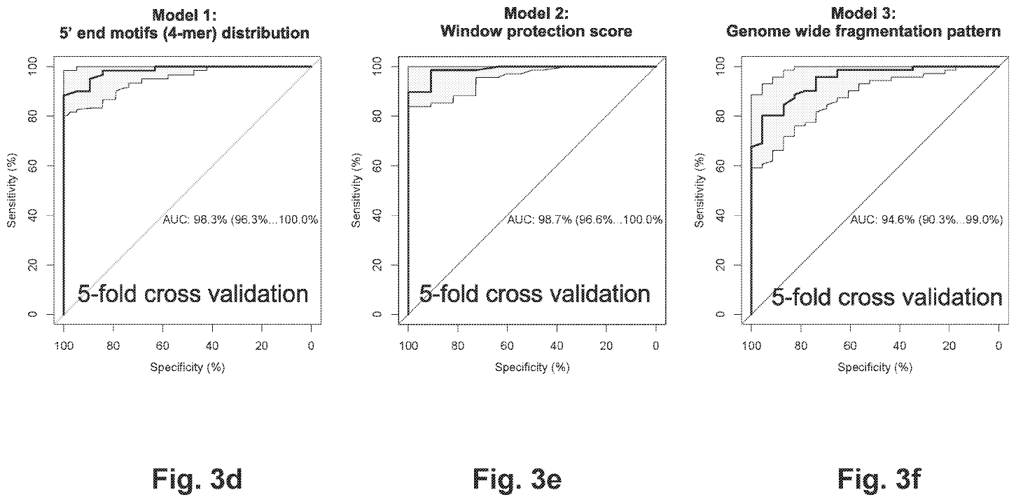

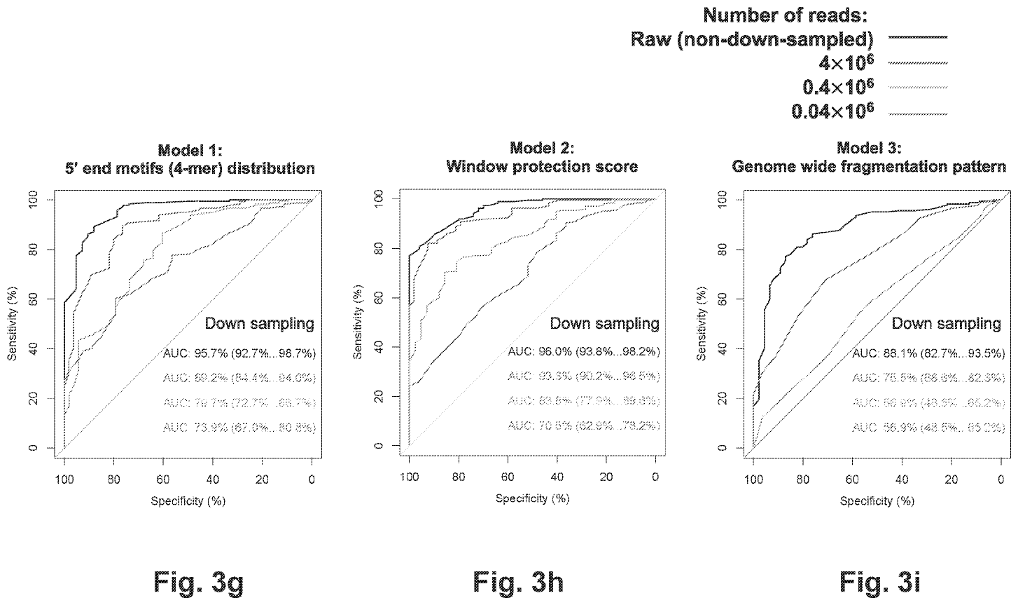

To achieve a more accurate cancer versus non-cancer classification, supervised learning of ctDNA fragmentation features was employed. Therefore, as described earlier, three random forest classifiers were trained each of which captured distinct features of targeted ctDNA fragmentation pattern. FIGS. 3a-f show three separate random forest classifiers trained and validated using ctDNA fragmentomics after 2-fold and 5-fold CV. Interestingly, all three models achieved high sensitivity and specificity in predicting cancer samples using 2- or 5-fold cross validation (CV). All three models showed similar performance to previously observed ctDNA cancer detection models using whole genome sequencing of ctDNA (FIGS. 3a-c)¹⁵⁻¹⁸. Namely, 2-fold CV achieved AUC more than 95% in model 1 (motif analysis) and model 2 (WPS) as compared to 88% in model 3 (GWFLD). A 5-fold CV overall improved the classification accuracy by 3% across all three models suggesting models trained on larger sample size can further boost the disclosed predictions (FIGS. 3d-f). It is worth noting that a lower AUC of model 3 might be a caveat of targeted approach as genome wide fragmentation pattern might be more sensitive to genomic window assessed. However, targeted ctDNA sequencing requires sequencing of substantially lower amount (by a factor of ˜30) of ctDNA compared to whole genome ctDNA suggesting GWFLD requires larger fragments compared than the other two models.

### Example 4

To compare the sequencing coverage needed to accurately predict cancer samples, random in silico down sampling of ctDNA fragments was performed and the performance of the three models evaluated. FIGS. 3g-i show the results of in silico down sampling of ctDNA sequencing reads that were performed to assess the sensitivity of the three models as a function of total number of reads used for each classification. Interestingly, leveraging only 4 million fragments can achieve near 90% sensitivity at 80% specificity in model 1 and 2 while model 3 required a higher sequencing coverage (FIGS. 3g-i).

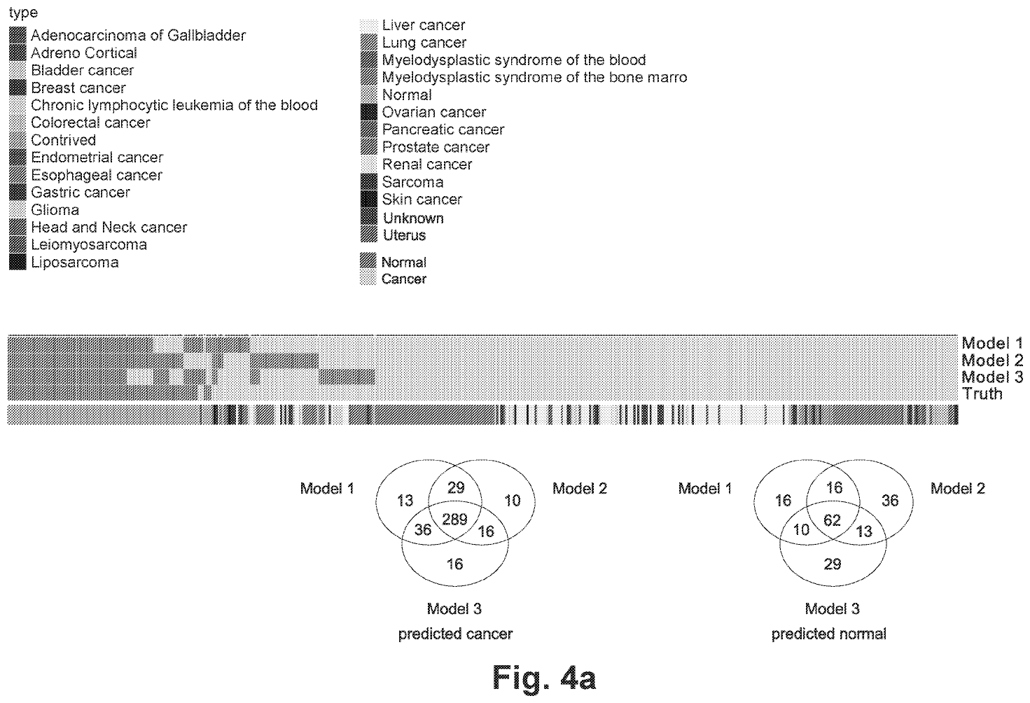

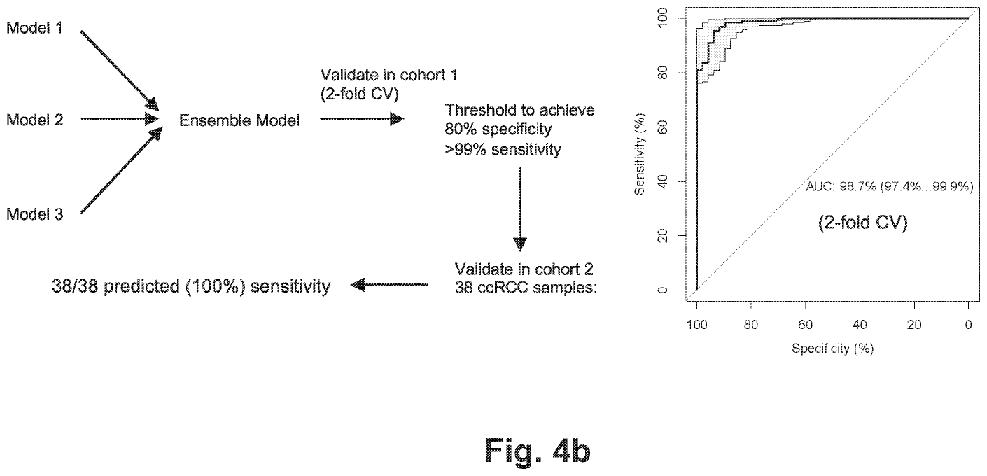

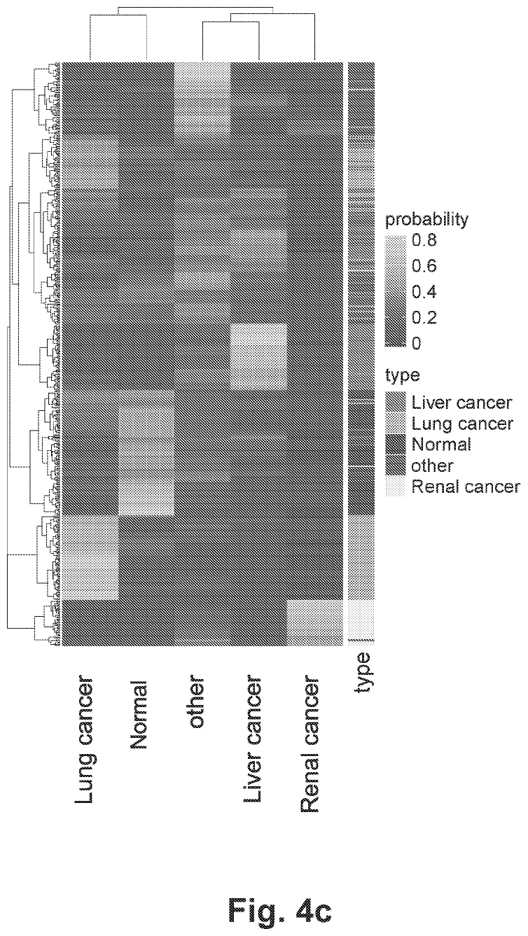

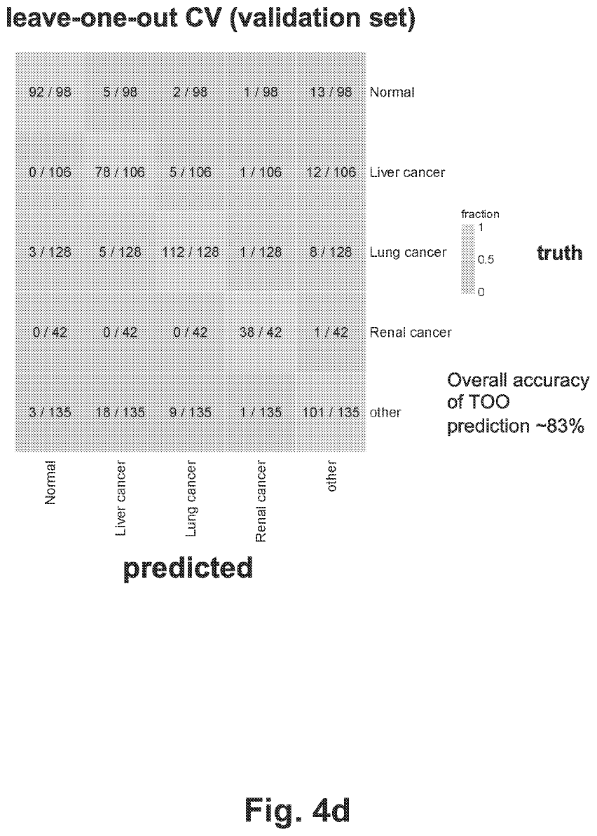

To leverage all fragmentomics features simultaneously, it was then sought to aggregate the predictions of the 3 models using an ensemble classifier (FIG. 4a). Thirty-eight out of 509 samples, comprising samples collected from a neoadjuvant immune checkpoint inhibitor therapy trial of patients with ccRCC by MSKCC¹⁹, were excluded in order to be used as an independent validation cohort (cohort 2). First, leave-one-out cross validation (LOO-CV) on the remaining 471 samples was performed to explore the concordance between the predictions made by the three models. Interestingly, the three models agreed in only 75% of cases suggesting an ensemble classifier might boost the performance of each individual classifier (FIG. 4a). As expected, an ensemble classifier that combined the predictions of the models, outperformed each individual model with an AUC=98.7% in a 2-fold CV. By setting a hard threshold for the random forest probability to obtain a binary class such that 80% specificity was achieved, the disclosed ensemble caller achieved ˜99% sensitivity in the disclosed validation set from a 2-fold CV. Strikingly, the same classifier achieved 100% sensitivity (38/38) in predicting cancer samples in an independent cohort (cohort 2) even though ccRCC has shown to be among the cancer types with the lowest ctDNA tumor fraction and requires a higher limit of detection²⁰ (FIG. 4b).

### Example 5

Finally, whether the disclosed ensemble classifier can be utilized to predict the TOO was explored, and LOO CV on all 509 samples was performed and each sample assigned a specific cancer type. Due to the small number of samples in certain cancer types, classifying lung cancer, liver cancer and renal cancer and combined other cancer types into a single subset as “other” was focused on. The disclosed ensemble classifier could predict the TOO with high accuracy (˜83%) demonstrating combining fragmentation features of targeted ctDNA can facilitate the prediction of cancer as well as TOO from circulating ctDNA (FIG. 4c).

### Example 6—cDNA Sequencing

The cell-free DNA was extracted from 2.0 ml-6.0 ml of plasma collected in Streck or EDTA blood tubes using the Qiamp Circulating Nucleic Acid Isolation Kit (Qiagen). The extracted cfDNA was quantified using the region quantified using a capillary electrophoresis-based method Fragment Analyzer (Agilent) and was between 75-250 bp. Libraries for ctDNA sequencing were generated with TruSight Oncology 500 ctDNA Library Prep Kit with 30 ng input of cfDNA material per sample. The workflow utilizes the same library preparation reagents as the TruSight Oncology 500 product for FFPE. Briefly, DNA fragments are end-repaired, A-tailed, and then ligated to adapters prior to sample barcoding via PCR. Unique molecular identifiers (UMIs) are incorporated into the adapters added via ligation and duplex barcodes added during PCR amplification were used for error correction. TruSight Oncology index PCR products were directly used for enrichment and libraries were enriched by hybrid-capture method. Enrichment of targeted regions required two hybridization steps at 57° C. AccuClear Ultra High Sensitivity dsDNA assay (Biotium) was used to ensure sufficient yield of the post-enriched libraries prior to normalization. Post enrichment libraries were normalized using bead-based normalization and pooled in equal volumes. Samples were sequenced with 151 bp paired-end reads on Illumina NovaSeq™ 6000 S4 flow cell using the XP workflow for individual lane loading (6-plex per lane, 24-plex per flowcell). On average, each sample yielded ˜1B reads per library.

### Example 7—Fragmentomics Feature Extraction

Illumina TruSight Oncology 500 ctDNA software was used to process sequencing reads. Aligned reads after adapter trimming, UMI collapsing and removing PCR duplicates were used to extract targeted ctDNA fragmentation features. Genome wide fragmentation pattern was generated as previously described¹⁶. Briefly, the ratio of short (100-150 bp) to long fragments (151-220 bp) was calculated across a 5 Mbp moving window across the genome. Then, GC normalization was performed to account for the bias in sequencing coverage followed by z-score normalization. WPS was calculated similar to previously described¹⁷ by measuring the fraction number of reads ending within a 120 bp moving window followed by coverage normalization. For motif analysis, the first 4 bases of each 5′-end from paired end reads were extracted generating 256 features corresponding to all possible 4-mers. Motifs containing low base quality or masked bases were excluded from the 4-mer set. 4-mer counts were normalized to account for the variability in sequencing depth. Motif diversity score (MDS) was calculated using Shannon Entropy index for each sample independent using the distribution of 256 4-mer motifs.

### Example 8—Construction of a Machine Learning Based Fragmentomics Classifier

Machine learning was used to distinguish cancer versus non-cancer samples using targeted ctDNA fragmentation pattern. Briefly, samples were randomly split into training and validation sets using 2- or 5-fold cross validation (CV) as stated. All trainings and validations were repeated 50 times to avoid any sampling bias in the disclosed analysis and confidence intervals were generated. To avoid overfitting, dimensionality reduction was performed on the training set using principal component (PC) analysis and top PCs were used i.e., top 30 PCs for motif analysis and top 40 PCs for GWFLD and WPS. For cancer versus non-cancer classification, 3 random forest binary classifiers were trained independently for each ctDNA fragmentation feature. To evaluate the performance of each classifier, each model was tested on the validation set after projecting features on the training set PCs. The area under the receiver operating characteristic (ROC) curve (AUC) was assessed using sensitivity and specificity as performance metrics. Next, an ensemble classifier was built that incorporates each of the three described random forest models into a single prediction by averaging the predicted probability of each possible outcome. For TOO classification, five groups were compared: normal tissue, lung, liver, renal cancer, and “Other” which contained other cancer types. Due to a relatively smaller sample size in each cancer type, an ensemble classifier was trained and tested using leave-one-out (LOO) CV on the 509 samples set and prediction accuracy was calculated as the ratio of correctly predicted TOO over the total number of samples for each cancer type.

### Terminology

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A. B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCES

1. Bray, F. et al. Global cancer statistics 2018: GLOBOCAN estimates of incidence and mortality worldwide for 36 cancers in 185 countries. CA: a cancer journal for clinicians 68, 394-424 (2018).
2. Liu, M. et al. Sensitive and specific multi-cancer detection and localization using methylation signatures in cell-free DNA. Annals of Oncology 31, 745-759 (2020).
3. Aravanis, A. M., Lee, M. & Klausner, R. D. Next-generation sequencing of circulating tumor DNA for early cancer detection. Cell 168, 571-574 (2017).
4. Phallen, J. et al. Direct detection of early-stage cancers using circulating tumor DNA. Science translational medicine 9(2017).
5. Wan, J. C. et al. Liquid biopsies come of age: towards implementation of circulating tumour DNA. Nature Reviews Cancer 17, 223-238 (2017).
6. Chin, R-I. et al. Detection of solid tumor molecular residual disease (MRD) using circulating tumor DNA (ctDNA). Molecular diagnosis & therapy 23, 311-331 (2019).
7. Abbosh, C., Birkbak, N.J. & Swanton, C. Early stage NSCLC-challenges to implementing ctDNA-based screening and MRD detection. Nature Reviews Clinical Oncology 15, 577-586 (2018).
8. Kang, S. et al. CancerLocator: non-invasive cancer diagnosis and tissue-of-origin prediction using methylation profiles of cell-free DNA. Genome biology 18, 1-12 (2017).
9. Keller, L., Belloum, Y., Wikman, H. & Pantel, K. Clinical relevance of blood-based ctDNA analysis: Mutation detection and beyond. British Journal of Cancer 124, 345-358 (2021).
10. Heitzer, E., Ulz, P., Geigl, J. B. & Speicher, M. R. Non-invasive detection of genome-wide somatic copy number alterations by liquid biopsies. Molecular oncology 10, 494-502(2016).
11. Lenaerts, L. et al. Genomewide copy number alteration screening of circulating plasma DNA: potential for the detection of incipient tumors. Annals of Oncology 30, 85-95 (2019).
12. Paracchini, L. et al. Genome-wide Copy-number alterations in circulating tumor DNA as a novel biomarker for patients with High-grade serous ovarian Cancer. Clinical Cancer Research 27, 2549-2559 (2021).
13. Guler, G. D. et al. Detection of early stage pancreatic cancer using 5-hydroxymethylcytosine signatures in circulating cell free DNA. Nature communications 11, 1-12 (2020).
14. Liu, Y. At the dawn: cell-free DNA fragmentomics and gene regulation. British journal of cancer, 1-12 (2021).
15. Mathios, D. et al. Detection and characterization of lung cancer using cell-free DNA fragmentomes. Nature communications 12, 1-14 (2021).
16. Cristiano, S. et al. Genome-wide cell-free DNA fragmentation in patients with cancer. Nature 570, 385-389 (2019).
17. Snyder, M. W., Kircher, M., Hill, A. J., Daza, R M. & Shendure, J. Cell-free DNA comprises an in vivo nucleosome footprint that informs its tissues-of-origin. Cell 164, 57-68 (2016).
18. Jiang, P. et al. Plasma DNA end-motif profiling as a fragmentomic marker in cancer, pregnancy, and transplantation. Cancer discovery 10, 664-673 (2020).
19. Krishna, C. et al. Single-cell sequencing links multiregional immune landscapes and tissue-resident T cells in ccRCC to tumor topology and therapy efficacy. Cancer Cell 39, 662-677. e6 (2021).
20. Zhang, Y. et al. Pan-cancer circulating tumor DNA detection in over 10,000 Chinese patients. Nature Communications 12, 1-14 (2021).
21. Serpas, L. et al. Dnase1l3 deletion causes aberrations in length and end-motif frequencies in plasma DNA. Proceedings of the National Academy of Sciences 116, 641-649 (2019).
22. Avgeris, M., Marmarinos, A., Gourgiotis, D. & Scorilas, A. Jagged Ends of Cell-Free DNA: Rebranding Fragmentomics in Modern Liquid Biopsy Diagnostics. (Oxford University Press, 2021).
23. Chan, K. A. et al. Second generation noninvasive fetal genome analysis reveals de novo mutations, single-base parental inheritance, and preferred DNA ends. Proceedings of the National Academy of Sciences 113, E8159-E8168 (2016).
24. Jiang, P. et al. Preferred end coordinates and somatic variants as signatures of circulating tumor DNA associated with hepatocellular carcinoma. Proceedings of the National Academy of Sciences 115, E10925-E10933 (2018).
25. Umetani, N. et al. Prediction of breast tumor progression by integrity of free circulating DNA in serum. Journal of clinical oncology 24, 4270-4276 (2006).
26. Wang, B. G. et al. Increased plasma DNA integrity in cancer patients. Cancer research 63, 3966-3968 (2003).
27. Kohabir, K., Wolthuis, R & Sistermans, E. A. Fragmentomic cfDNA Patterns in Noninvasive Prenatal Testing and Beyond. Journal of Biomedicine and Translational Research 7(2021).
28. Chiu, R W., Heitzer, E., Lo, Y. D., Mouliere, F. & Tsui, D. W. Cell-free DNA fragmentomics: the new “Omics” on the block. Clinical Chemistry 66, 1480-1484 (2020).

What is claimed is:

1. A method of detecting a cancer in a subject, the method comprising:
obtaining a sample from a subject;
isolating cfDNA from the sample;
generating sequence libraries of ctDNA fragments from the isolated cfDNA;
analyzing the sequence libraries of the ctDNA fragments to identify a tissue of origin of the ctDNA,
thereby detecting the cancer in the subject.

2. The method of claim 1, wherein analyzing the sequence libraries of ctDNA fragments comprises analyzing 5′end 4-mer motifs in the ctDNA fragments.

3. The method of claim 2, wherein analysis of 5′end 4-mer DNA motifs in the ctDNA fragments comprises an unbiased enrichment analysis of one or more motifs associated with a cancer tissue of origin as compared to an expected distribution of the one or more motifs in a healthy tissue of origin.

4. The method of claim 3, wherein the method achieves an AUC of at least 95% in detecting the cancer.

5. The method of claim 1, wherein analyzing the sequence libraries of the ctDNA fragments comprises performing a window protection score (WPS) analysis.

6. The method of claim 5, wherein the window protection score analysis comprises determining a ratio of a number of endpoints of the ctDNA fragments within a 120 bp ctDNA fragment size window to a number of fragments completely spanning the 120 bp ctDNA fragment size window.

7. The method of claim 6, wherein a high WPS value as compared to a threshold indicates an increased protection of the ctDNA from digestion.

8. The method of claim 6, wherein a low WPS value as compared to a threshold indicates a decreased protection of the ctDNA from digestion.

9. The method of claim 6, wherein the method achieves an AUC of at least 95% in detecting the cancer.

10. The method of claim 1, wherein analyzing the sequence libraries of the ctDNA fragments comprises performing a genome wide fragmentation length distribution (GWFLD) analysis.

11. The method of claim 10, wherein the GWFLD analysis comprises determining a ratio of a number of ctDNA fragments that range in size from greater than 100 to less than 150 bp to a number of ctDNA fragments that range in size from greater than 151 to less than 220 bp within a 5 Mbp ctDNA fragment size window.

12. The method of claim 11, wherein the method achieves an AUC of more than 90% in detecting the cancer.

13. The method of claim 1, wherein generating sequence libraries of ctDNA fragments comprises targeted enrichment of 100 or more, 200 or more, 300 or more, 400 or more, 500 or more genomic regions.

14. A method of identifying a tissue of origin of cfDNA in a subject, the method comprising:
obtaining a sample from a subject;
isolating cfDNA from the sample;
generating sequence libraries of ctDNA fragments from the cfDNA;
aligning paired-end reads of the ctDNA fragments;
analyzing 5′end 4-mer motifs, window protection score (WPS), and genome wide fragmentation length distribution (GWFLD) of the ctDNA fragments;
generating independent data models for 5′end 4-mer motifs, WPS, and GWFLD;
generating an ensemble model of the independent models from the independent data models using a machine learning process; and
classifying the sample as comprising ctDNA fragments from a healthy tissue or a cancer tissue based on the ensemble model.

15. The method of claim 14, wherein the method provides at least 80% sensitivity at 99.9% specificity in classifying the sample as comprising ctDNA from a healthy tissue or a cancer.

16. The method of claim 14, wherein the method provides at least 80% accuracy in predicting a tissue of origin of the ctDNA fragments.

17. The method of claim 14, wherein generating sequence libraries of ctDNA fragments comprises targeted enrichment of 100 or more, 200 or more, 300 or more, 400 or more, 500 or more genomic regions.

18. A method of determining a presence of cancer in a patient, the method comprising:
isolating cfDNA from samples from a plurality of subjects;
generating sequence libraries of ctDNA fragments from the isolated cfDNA;
aligning paired-end reads of the ctDNA fragments;
analyzing 5′end 4-mer motifs, window protection score (WPS), and genome wide fragmentation length distribution (GWFLD) of the ctDNA fragments;
generating independent models for 5′end 4-mer motifs, WPS, and GWFLD;
generating an ensemble model of the independent models using a machine learning process from the independent models;
applying the ensemble model to sequence libraries of ctDNA fragments for a sample obtained from the patient; and
determining whether the patient has cancer based on the application of the ensemble model.

19. The method of claim 18, wherein generating sequence libraries of ctDNA fragments comprises targeted enrichment of 100 or more, 200 or more, 300 or more, 400 or more, 500 or more genomic regions.

20. The method of any one of the foregoing claims, wherein the sample is blood.

21. The method of any one of the foregoing claims, wherein the cancer is one or more of lung cancer, liver cancer, renal cancer, breast cancer, glioma, or colorectal cancer.

22. The method of any one of the foregoing claims, wherein generating sequence libraries of ctDNA fragments comprises enriching, isolating, and sequencing a subset of genes or genomic region of interest.

23. The method of any one of the foregoing claims, wherein enriching, isolating, and sequencing a subset of genes or regions of a genome comprises capturing a subset of genes or genomic region of interest by hybridization of ctDNA fragments to probes that are specific for the subset of genes or genomic region of interest.
